SEQUENCE LISTING (19 sequences)

SEQ ID NO: 1 - protein, 136 aa, Corynebacterium glutamicum
MLRTILGSKIHRATVTQADLDYVGSVTIDADLVHAAGLIEGEKVAIVDITNGARLETYVIVGDAGTGNICINGAAAHLINPGDLVIIMSYLQATDAEAKAYEPKIVHVDADNRIVALGNDLAEALPGSGLLTSRSI

SEQ ID NO: 2 - protein, 930 aa, Gallus gallus
MISVDRLSEEQALGMKEQEWAGPEALCPGWQEEEVSDGEGPEDSGHPDPTAHAYEVLQHTLRLEGMPLTIDRTGQPRTGSGPLDMTVCVLGSPTAFLPVLLEGGTRYPGAMVLCLAPAWASRVPSETSPGSWSLLLSRGVSFEAGGCTALEEFVPPRRATYVTGTFGSEGSWEGELARDLDCPTGGSALLTRWLEDPLLSRWLLSARAGLPVPPTLAFITGLWETLPEEPEPPGVHLVRLQDPQGQESLVRDEVGAFLEGSSMQPYDQVAVRLSGWRWRGTDPHSTHRKVEGEAVAQAVAALLKGLREEESILLEALVPTARLPTLPPRSAAPRLPMALRICTVVCRSWGDRPQLCQVACTAGRAEVPVRHGSALPLGLDSSLRQWGLADAAQRQALAGQLREAAEAAMAALLAAEGELSPAQRGGARAHTDVLGVDFLLACVDGTLELVALSANCLRCLETCLLAEGMGHDVGQPAGDVPRLLAECLLHRAQCHLVEGKDILLIGAGGVSKSFVWEAAREYGLRIHLVESDPEHFAAGLVETFLPYDSREHRRDEEHAERVLEMLRARGLRPDACLSYWDDCVVLTALLCQRLGLPGCPPAAVRLAKQKSRTHQHLQRCRRGRPPPAAFSVPCRRLRSHGDVERAAGAVPFPAVAKLEFGAGAVGVRLVENAGQCHAHAAQLWHDLRADADHPGIGLGWGNAMLLMEYVPGTEHDVDLVLFEGRLLGAWVSDNGPTRVPTFLETAATLPSCLPADRQAQLVRAALRCCRACGLRHGVFNVELKLSPAGPRLLEINPRMGGFYLRDWMRAVYGPDLLLAAVLLALGLPPVLPSRPAPRQQLAGVMCLASEHGRALRGGVMAALQGLQRRGLVRLNPLFEEAGGRYEEPCLSVACAGDGPAEACGRLLGLCQALGIDSPQYPVGHFLSHFK

SEQ ID NO: 3 - protein, 127 aa, Escherichia coli
MKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDRGVMTAFMQALGFTAQQGGWEKC

SEQ ID NO: 4 - protein, 221 aa, Acropora millepora
MSVIAKQMTYKVYMSGTVNGHYFEVEGDGKGKPYEGEQTVKLTVTKGGPLPFAWDILSPQCQYGSIPFTKYPEDIPDYVKQSFPEGYTWERIMNFEDGAVCTVSNDSSIQGNCFIYHVKFSGLNFPPNGPVMQKKTQGWEPNTERLFARDGMLLGNNFMALKLEGGGHYLCEFKTTYKAKKPVKMPGYHYVDRKLDVTNHNKDYTSVEQCEISIARKPVVA

SEQ ID NO: 5 - protein, 950 aa, Gorilla gorilla
MLSLDPSGPEWDCPLGSKDLEEEGPWGGGSGLPPTGCFPGSWRQDVGLDCKGSPEGAEARAWTVYYYSLLQSCLQQAGLPETQDRSQVPRTGCPGAEVTLCVLGSPSTFLPVLLEGGVQSPGNMLLCLSPAWLMKVPAPGQPGEAALLVSKAVSFHPGGLTFLDDFVPPRRATYFLAGLGPGPGRGREAAELARDLTCPTGASAELARLLEDRLLTRQLLAQQGGVAVPATLAFTYKPPGLLLGGDASLGLRLVELSGKEGQEMLVKEEVEAFLRSEALGDILQVAVKLSGWRWRGRQAWRLHPRAELGAVVDTVLALLEKLEEEESVLVEAVYPPAQLPCSDGPSPGPGLAVRICAVVCRIQGDRPLLSKVVCGVGRGDRPLRHHNSLPRTLEVALAQCGLGEEAQVAAVRQRVKAAAEAALAAVLALEAGLSAEQRGGRRAHTDFLGVDFALTAAGGVLTPVALELNGGLCLEACGALEGLWAAPRLGPAADEAVAAPLVETMLRRSARCLMEGKQLLVVGAGGVSKKFVWEAARDYGLQLHLVESDPNHFASQLVQTFIHFDMTEHRRDEENARLLAELVRARGLKLDGCFSYWDDCLVLTALLCQELGLPCSSPAAMRLAKQKSLTQLHLLRHHGPPWPAPSLHAVPCCPLESEADVERAVHQVPLPGVMKLEFGAGAVGVRLVEDAPQCHEHFSRITRDLQGEADHPGIGLGWGNAMLLMEFVEGTEHDVDLVLFGGRLLAAFVSDNGPTRLPGFTETAACMPTGLAPEQEAQMVQAAFRCCLGCGLLDGVFNVELKLTGAGPRLIEINPRMGGFYLRDWILELYGVDLLLAAVMVACGLRPALPTRPRARGHLVGVMCLVSQHLQALSSTASRETLQALHDRGLLRLNLLEEALVPGEYEEPYCSVACAGPSPIEARLRLLGLCQGLGIDGPSYPVAHFLSHFK

SEQ ID NO: 6 - protein, 777 aa, Falco peregrinus
MSAEPAPSPKEQEWAGPEALCPGWLEDEAPDGEMPEDSGDPDCATHAYELLQTALLQEGLPHTLDCSAEPRTGFGPLDMTVCILGSPTAFLPILLEGGTRCPGAMVLCLSPTWASRVPSETSPGAWSLLLSRGISFKEGGHSTLETFTPPRRANYVTGTFATGGSDGGWVGELARDLDCPTGGSVPLTRRLEDPLVTRWVLAARAGLPVPPTLAFVLGPGGHLPTDPVAPRVRLVRLEDPQGQESLVQEEVGAFLGGTAMEPYSQVLVRLAGWRWRGTGAHSTHGKEEGAAVAQAVGARLRRLREEDSVLLEALVPTARLPMPPPRSPAPRLPVALRICTLVCRSWGDRPQLCQVACGVGRAEAPVRHGAALPQGLDSSLQQWGGGAPRPRQGPGPRTAVATRRRGAAEAAMAALLAAEAELSPQQHGGTRARTNLLGVDFLLACVDGTLELVALSTNSQRCLETCLLAEAMGRAVGEPPGDLPRLLAEALLYQAQCHLVEGKDILLIGAGGVSKSFVWEAARDYGLRIHLVESDPEHFAAGLVQTFLPYDSREHRRDEEHAERVMELVQARGLRPHAAAVRVAKQKSRTHQHLQRCRRGRPPPATFAVPCCRLQSHGDVERAASAMPFPAVAKLEFGAGGVGVRLVESAGQCHAHAARLWRDLRDDADHPGIGLGWGNAMLLMEYVPGTEHDVDLVIFEGRLLGAWVSDNGPTRLPAFLETAACLPSCLPADRQGQLGRGAPSEGSAATVQDSTEPDSDSQPLRCLRAYNNTPYSA

SEQ ID NO: 7 - protein, 951 aa, Alligator mississippiensis
MLSLDQINVDQPLILKELDWAEQGPLPSIGSLRPHWRQDVSLDCKISPEDSHARACTAYYYDLLQGTLQQEGLPETIDRTKEPCTGFSSTEVTICILGSPTSYLSVLLEGGSQCPGSMLLCLSPCWLSKVPSLLRPGESSLLVSKAISFEQGGRTFLEEFSPPRRVTYFTGSFGPCKEHEQSAGELARDLDCPTGGSGELARLLEDKLLTRQLLDARAQVGVPPTLAFTFQRPQLLQGLAAQRSLHVVELSGKEGQENLVQEEVGAFLQGGAMEPYSQVVVKPSGWRWSGAHTVSFHVKAEQAAVLQAVLALLETLEEEESALVEAFIPTARFTQFSSPDNSSLCSTSPRPNLAIRICTVVCRSWADQPLLTKVVCGMGRADKPLKHQATVPQTLESSLQEWGMTDEAQITAIRAQIKQKAEAAMRAFMEMEAELSVEQRGGRRTQTDVIGVDFLLTSSEEVLQLVALEMNSQLCLETCALFESMGQAVGVPRGSSQPLVETMLRRAQCHLMEGKHVLVIGAGGVSKKFVWEAAREYGLKIHLVESDPNHFASQLVQTFIHYDSTDHKRDEEHAQRVVELVQERGLHLDGCLSYWDDCMVLTALVCEQLGLRCSPTAAMRVAKQKSRTHQHLLRRRKDAPRGWPSAAVYAVPCWHLESQADVERAAHHLSFPGVMKLEYGAGAVGVKLVEDAQQCHLHFEKISQDLREDTDHPGIGLGWGNAMLLMEYVSGTEHDVDLVIFEGRMLAAFVSDNGPTRVPCFTETAASMPTCLPPDREAQLVRAAYQCCLGCGLTDGVFNVELKLTAAGPKLIEINPRMGGFYLRDWIREVFGVDIMLAAVMVACGVAPLLPTWPQPRTHLVGVMCVVSQHMQALKSTASLETLQALHEGGVVRLNLLDDKLISREYEEPYCNVACGGPSRHEACLKVLSICQALGIDSPQYPVAHFLSHFK

SEQ ID NO: 8 - protein, 852 aa, Ailuropoda melanoleuca
MLSLDPLGPEWDCPLGSKDLEEEEGPWGGGSGLPPPGSFSGSWRHDVGLDCKGSLEGAEARAWTTYYYSLLQSCLQQAGLPETQDRSQVPRTGCPGAEVTLCILGSPSTFLSVLLEGGVQSPGNMLLCLSPAWLTKVPAPGQPGEAVLLVSKAVSFHPGGLTFLEDFVPPRRATYFLAGLGLGPGRGREAAELARDLTCPTGASAELARLLEDRLLTRRLLAQQGGVAVPATLAFTYKPPALLRGGDASPGVRLVELSGKEGQEALVKEEVGAFLHSGALGDALQVAVKLSGWRWRGRQALRVYPRVELGTVVDTVLALLEKLEEEESVLVEAVCPPARLPFPGSPPPGPELAVRICAVVCRTQGDRPLLSKVVCSVGRQDRPLRHRSSLPQTLEVALARCGLGETGQVAVVRQRVKTAAELLIGAGGVSKKFVWEAARDYGLKLHLVESDPNHFASQLVQTFIHFDVTEHRRDEENARLLAELVRARGLQLDGCFSYWDDCLVLTALLCQELGLPCSPPAAMRLAKQKSCTQLHLLRCQGPPWPAPSLHAVPCCPLESEADVEKAVHQVPLPGVMKLEFGAGAVGVRLVEDAPQCHEHFSRVARDLQGEADHPGIGLGWGNAMLLMEFVEGTEHDVDLVLFGGRLLAAFVSDNGPTRLPGFTETAACMPTGLAPEQEAQLVQAAFRCCLGCGLLDGVFNVELKLTGAGPKLIEINPRMGGFYLRDWILELYGVDLLLAAAMVACGLRPALPTHPRARGHLVGVMCLMSQHLQVLNSTSSRETLQGLHDQGLLRFNLLEEILEQGEYEEPYCSVACAGSSPAEARLRLLGLCQGLGIDGPDYPVAHFLSHFK

SEQ ID NO: 9 - protein, 827 aa, Ursus maritimus
MLSLDPLGPEWDCPLGSKDLEEEEGPWGGGSGLPPPGSFPGSWRQDVGLDCKGSLEGVEARAWTVYYYSLLQSCLQHAGLPETQDRSQVPRTGCPGAEVTLCILGSPSTFLSVLLEGGVQSPGNMLLCLSPAWLTKVPAPGQPGEAVLLVSKAVSFHPGGLTFLEDFVPPRRATYFLAGLGLGPGQGREAAELARDLTCPTGASAELARLLEDRLLTRRLLAQQGGVAVPATLAFTYKPPALLRGGDASPGVRMVELSGKEGQETLVKEEVGAFLHSGALGDALQVAVKLSGWRWRGRQALRVCPRAELGTVVDTVLALLEKLEEEESVLVEAVCPPARLPFPGSPPPGPELAVRICAVVCRTQGDRPLLSKVVCSVGRQDRPLRHRSSLPQTLEVALARCGLGETGQVAVVRHWWFLQLHLVESDPNHFASQLVQTFIHFDVTEHRRDEENARLLAELVRARGLQLDGCFSYWDDCLVLTALLCQELGLPCSPPAAMRLAKQKSCTQLHLLRCQGPPWPAPSLHAVPCCPLESEADVEKAVHQVPLPGVMKLEFGAGAVGVRLVEDAPQCHEHFSRVARDLQGEADHPGIGLGWGNAMLLMEFVEGTEHDVDLVLFGGRLLAAFVSDNGPTRLPGFTETAACMPTGLAPEQEAQLVQAAFRCCLGCGLLDGVFNVELKLTGAGPKLIEINPRMGGFYLRDWILELYGVDLLLAAAMVACGLRPALPTHPRARGHLVGVMCLMSQHLQVLNSTSSRETLQALHDQGLLRFNLLEEILEQGEYEEPYCSVACAGSSPAEARLRLLGLCQGLGIDGPHYPVAHFLSHFK

SEQ ID NO: 10 - protein, 954 aa, Python bivittatus
MLSLDQIHTEPPQTPKELDWAEQSFLPSLSPLRSAWHRDTLHDSKVNPEGAQARACTAYYYDLLQCSLHQKGLPGTIDLTKEPRSGFRPSDITICVLGSPASYLSVLLEGGNQCPGNMLLCLSSTWLSKVPSETHPGESSLFVLKAVTFELGGRTVLEEFSSPRRVNYFTGSFGPCKVQGQSAAELARDLDCPTGGSGELTRLLEDKLLTRQLMDQKGEVAVPPTLAFTFKRPLLLHNVTEEQRIHVVELSGREGQDNLLQEEITAFLQGGAMESYKQVVVKPSGWRCSGTQAVSFLDKSEHGSVLQAVLTLLETLEEEESILLEPFIPTAHIIQSGPSNTNSSPVPGNISLRPNLAIRICAVVCRSRKDQPLLSKVICGVGPTNKPLKHQTTVPQTLESSLRHWGLKDEVQIAAIHAQIKQKAEAAMKAFMDMETSLSAEQKGGCRAQTDIIGVDFLLSSEDQVLQLVALEMNSQLCLETCSVLETMGRMVGAPGGETACALVETMLRRAQCHLMEGKHVLVIGAGGFSKKFVWEAARDYGLKIHLVETDPNHFASQLVHTFIHYDTTDHKKDEEHAQALLGLIQERGLHLDGCLSYWDDCMVLTALVCEGLGLRCSSSTAMRVAKQKSRTHQHLLRRCKEGSRWSSAALYAVPCCHLESHADVERAASHINFPGVMKLEFGAGAVGVKLVEDAQQCHLHFDKISHDLQDDSDYLGIGLGWGNTMLLMEYVSGTEHDVDLVIYEGRMLGAFVSDNGPTRVPNFTETAACMPTCLPPDCEAQLIHAAYQCCLGCGLTDGVFNVELKLTAAGPKLIEINPRMGGFYLRDWIQEIYGVDIMLASVMVACGVTPVLPTRSCPRTNLVGVMCLVSQHLQALKSTASLETLQALHERGVIRLNLLEDEIVSKEYEEPYCNVACASSSRQEACLKLLGICQVLGIDSPHYPVAHFLSHFK

SEQ ID NO: 11 - protein, 827 aa, Orcinus orca
MLLCLSPAWLTKVPAPGQPGEAALLVSKAVSFHPGGLTFLDDFVPPRRATYFLAGLGLGPSRGREAAELARDLTCPTGASAELARLLEDRLLTRRLLARQGDVAVPATLAFTYKPPALLRGGDASPGLRLVELNGKEGQETLVKEEVGAFLHSEALGDALQVAMKLSGWRWRGRQAMRLYPRAELGPVVDTVLALLEKLEEEESVLVEAVCPPARLPFPGSPLPGPELALRICAVVCRTQGDKPLLSKVVCSVGRGDRPLRHQSSLPRTLEAALAQCGLGEASQVAVVRQRVKAAAEAALAAVLTLEASLSAEQRGGRQVRTDFLGVDFALTVAGRALTPVALELNGGLCLEACGALEGLWAAPRPGLAAEEAAAAPLVETMLRRSARCLMEGKQLLLLGAGGVSKKFVWEAARDYGLKLHLVDSDPNHFASQLVQTFIHFNITEHQRDEENARVLAELVRARGLQLDGCFSYWDDCLVLTALLCQELGLPCNPPAAMHVAKQKSRTQLHLLCHHGPPWPAPSLYAVPCCPLESEADVDRAVRQVPLPGVMKLEFGAGAVGVRLVEDASQCHEHFSRIARDLQVEADHPGIGLGWGNAMLLMEFIEGTEHDVDLVLFGGRLLAAFVSDNGPTRLPGFTETAACMPTGLAPEQEAQLVQAAFRCCLGCGLLDGVFNVELKLTRAGPRLIEINPRMGGFYLRDWILELYGVDLLLAAAMVACGLRPALPAHPRARGHLVGVMCLVSQHLQALSSTASRETLQALHDQGLLRLNLLEETLVPGEYEEPYCSVACAGPSPAEARLRLLGLCQGLGIDGPHYPVAYFLSHFK

SEQ ID NO: 12 - protein, 126 aa, Escherichia coli
MIRTMLQGKLHRVKVTHADLHYEGSCAIDQDFLDAAGILENEAIDIWNVTNGKRFSTYAIAAERGSRIISVNGAAAHCASVGDIVIIASFVTMPDEEARTWRPNVAYFEGDNEMKRTAKAIPVQVA

SEQ ID NO: 13 - protein, 396 aa, Methanocaldococcus jannaschii DSM 2661
MRNMQEKGVSEKEILEELKKYRSLDLKYEDGNIFGSMCSNVLPITRKIVDIFLETNLGDPGLFKGTKLLEEKAVALLGSLLNNKDAYGHIVSGGTEANLMALRCIKNIWREKRRKGLSKNEHPKIIVPITAHFSFEKGREMMDLEYIYAPIKEDYTIDEKFVKDAVEDYDVDGIIGIAGTTELGTIDNIEELSKIAKENNIYIHVDAAFGGLVIPFLDDKYKKKGVNYKFDFSLGVDSITIDPHKMGHCPIPSGGILFKDIGYKRYLDVDAPYLTETRQATILGTRVGFGGACTYAVLRYLGREGQRKIVNECMENTLYLYKKLKENNFKPVIEPILNIVAIEDEDYKEVCKKLRDRGIYVSVCNCVKALRIVVMPHIKREHIDNFIEILNSIKRD

SEQ ID NO: 14 - protein, 117 aa, Helicobacter pylori
MTFEMLYSKIHRATITDANLNYIGSITIDEDLAKLAKLREGMKVEIVDVNNGERFSTYVILGKKRGEICVNGAAARKVAIGDVVIILAYASMNEDEINAHKPSIVLVDEKNEILEKG

SEQ ID NO: 15 - protein, 540 aa, Tribolium castaneum
MPATGEDQDLVQDLIEEPATFSDAVLSSDEELFHQKCPKPAPIYSPVSKPVSFESLPNRRLHEEFLRSSVDVLLQEAVFEGTNRKNRVLQWREPEELRRLMDFGVRSAPSTHEELLEVLKKVVTYSVKTGHPYFVNQLFSAVDPYGLVAQWATDALNPSVYTYEVSPVFVLMEEVVLREMRAIVGFEGGKGDGIFCPGGSIANGYAISCARYRFMPDIKKKGLHSLPRLVLFTSEDAHYSIKKLASFQGIGTDNVYLIRTDARGRMDVSHLVEEIERSLREGAAPFMVSATAGTTVIGAFDPIEKIADVCQKYKLWLHVDAAWGGGALVSAKHRHLLKGIERADSVTWNPHKLLTAPQQCSTLLLRHEGVLAEAHSTNAAYLFQKDKFYDTKYDTGDKHIQCGRRADVLKFWFMWKAKGTSGLEKHVDKVFENARFFTDCIKNREGFEMVIAEPEYTNICFWYVPKSLRGRKDEADYKDKLHKVAPRIKERMMKEGSMMVTYQAQKGHPNFFRIVFQNSGLDKADMVHLVEEIERLGSDL

SEQ ID NO: 16 - protein, 393 aa, Methanocaldococcus bathoardescens
MQEKGVSEREILEELIKYRDLDLKYEDGKIFGSMCSNILPITRKIVDMFLETNLGDPGLFKGTKLLEEKAIALLGSLLNNKNAYGHIVSGGTEANLMALRCIKNIWREKKRKGLSKNERPKIIIPVTAHFSFEKGRDMMDLDYIYAPIKKDYTIDEKFVRDAVEDYEIDGIIGIAGTTELGTIDNIEELSKIAKENDIYIHVDAAFGGFVIPFLEDKYKKKGVNYKFDFSLGVDSITIDPHKMGHCPIPSGGILFKDMSYKKYLDVNAPYLTETRQATILGTRVGFGGACTYAVLRYLGREGQRKIVSECMENTLYLYKKLKENNFKPVIEPILNIVAIEDEDYKEICKKLRDRGIYVSVCNCVKALRIVVMPHIKKEHIDNLIETLKIIKKD

SEQ ID NO: 17 - protein, 126 aa, Pectobacterium carotovorum
MIRTMLQGKLHRVKVTQADLHYEGSCAIDQDFMDAAGILEYEAIDIYNVDNGQRFSTYAIAGERGSRIISVNGAAARLACVGDKLIICSYVQMSDEEARSHSPKVAYFSGDNEMQRQAKAIPVQVA

SEQ ID NO: 18 - protein, 142 aa, Actinoplanes sp. SE50/110
MLRTMLKSKIHRATVTQADLHYVGSVTIDEDLLEAADLLPGEQVAIVDVTNGARLETYVIPGERGSGVIGINGAAAHLVHPGDLVILISYGQMDDAEAREYRPRVVHVDAQNRVIELGADPAEAVPGMAGDLVRGDLTLATR

SEQ ID NO: 19 - protein, 126 aa, Raoultella ornithinolytica
MIRNMLQGKLHRVKVTQADLHYEGSCAIDQDFLDASGILENEAIHIWNVTNGNRFSTYAIAAERGSRIISVNGAAAHCASVGDILIIASFVTMPDEEARRWQPKVAYFEGDNEMKRQAKAIPVQVA

(19) United States
(12) Patent Application Publication
(10) Pub. No.: US 2017/0211105 A1
(43) Pub. Date: Jul. 27, 2017

(54) BIOSYNTHETIC PRODUCTION OF CARNOSINE AND BETA-ALANINE

(71) Applicant: 20n Labs, Inc., Washington, DC (US)

(72) Inventors: John Christopher Anderson, Berkeley, CA (US); Saurabh Srivastava, San Francisco, CA (US); Mark T. Daly, Oakland, CA (US)

(21) Appl. No.: 15/408,317

(22) Filed: Jan. 17, 2017

Related U.S. Application Data
(60) Provisional application No. 62/281,621, filed on Jan. 21, 2016.

(51) Int. Cl. C12P 13/00; C12P 13/06; C12R 1/865

(52) U.S. Cl. CPC C12P 13/005; C12R 1/865; C12P 13/06

(57) ABSTRACT

The present disclosure provides compositions and methods for the biosynthetic production of carnosine and beta-alanine.

### CROSS REFERENCE TO RELATED APPLICATIONS

This application claims priority to, and benefit of U.S. Provisional Application No. 62/281,621 filed Jan. 21, 2016, the contents of which are incorporated herein by reference in its entirety.

### INCORPORATION-BY-REFERENCE OF SEQUENCE LISTING

The contents of the text file named “NLAB_002_01US_ST25.txt” submitted electronically herewith which was created on Jan. 5, 2017 and is 80 KB in size, are incorporated herein by reference in their entirety.

### FIELD OF THE INVENTION

The present disclosure relates compositions and methods for the biosynthetic production of nutritional supplements such as beta-alanine and carnosine. In particular, the disclosure features recombinant microorganisms comprising an engineered carnosine biosynthesis pathway.

### BACKGROUND OF THE INVENTION

Carnosine is a dipeptide of the amino acids beta-alanine and histidine. It is highly concentrated in muscle and brain tissues.

β-Alanine (or beta-alanine) is a naturally occurring beta amino acid, which is an amino acid in which the amino group is at the β-position from the carboxylate group (i.e., two atoms away).

β-Alanine is not used in the biosynthesis of any major proteins or enzymes. It is formed in vivo by the degradation of dihydrouracil and carnosine. It is a component of the naturally occurring peptides carnosine and anserine and also of pantothenic acid (vitamin B5), which itself is a component of coenzyme A. Under normal conditions, β-alanine is metabolized into acetic acid.

β-Alanine is the rate-limiting precursor of carnosine, which is to say carnosine levels are limited by the amount of available β-alanine, not histidine. Supplementation with β-alanine has been shown to increase the concentration of carnosine in muscles, decrease fatigue in athletes and increase total muscular work done.

Carnosine and beta-alanine are popular dietary supplements currently produced using chemical methods. Beta-alanine is also a synthetic precursor to pantothenic acid, the essential vitamin B5. Beta-alanine can also be used as a monomer for the production of a polymeric resin (U.S. Pat. No. 4,082,730).

Naturally, carnosine is produced exclusively in animals from beta-alanine (via uracil) and histidine. In yeasts and animals, beta-alanine is typically produced by degradation of uracil. Chemically, carnosine can be synthesized from histidine and beta-alanine derivatives. For example, the coupling of an N-(thiocarboxy) anhydride of beta-alanine with histidine has been described (Vinick et al. A simple and efficient synthesis of L-carnosine. J. Org. Chem, 1983, 48(3), pp. 392-393).

Beta-alanine can be produced synthetically by Michael addition of ammonia to ethyl- or methyl-acrylate. This requires the use of the caustic agent ammonia and high pressures. It is also natively produced in bacteria and yeasts in small quantities. In bacteria, beta-alanine is produced by decarboxylation of aspartate. Lysates of bacteria have been used in biocatalytic production from aspartate (Patent CN104531796A).

There remains a need in the industry for a safer, more economical system for the production of carnosine and beta-alanine.

### SUMMARY OF THE INVENTION

The present disclosure provides compositions and methods for the biosynthetic production of nutritional supplements such as beta-alanine and carnosine.

Embodiments of the present invention comprise engineered organisms that produce beta-alanine and carnosine. The engineered organisms may include genetically tractable organisms such as plants, animals, bacteria, or fungi.

The present invention comprises methods of producing carnosine. The methods comprise providing a recombinant microorganism comprising an engineered carnosine biosynthesis pathway. The engineered microorganism may be used for the commercial production of carnosine. Accordingly, in one embodiment the invention provides growing in suitable conditions, a recombinant microbial host cell comprising at least one DNA molecule encoding an enzyme(s) that catalyze a substrate to product conversion selected from the group consisting of:

i. aspartate to beta-alanine (pathway step a);

ii. beta-alanine to carnosine (pathway step b);

the at least one DNA molecule is heterologous to said microbial host cell and wherein said microbial host cell produces carnosine. The method further includes cultivating the microorganism in a culture medium until a recoverable quantity of carnosine is produced and recovering the carnosine.

In one aspect, a biotransformation method of producing carnosine is provided. The method comprises providing a recombinant microorganism comprising an engineered carnosine synthesis pathway. The engineered microorganism may be used for the commercial production of carnosine. Accordingly, in one embodiment, the invention provides growing in suitable conditions, a recombinant microbial host cell comprising at least one DNA molecule encoding an exogenous enzyme that catalyzes the joining of beta-alanine to histidine to produce carnosine wherein the at least one DNA molecule is heterologous to said microbial host cell, wherein beta-alanine substrate is added to the growth culture, and wherein said microbial host cell produces carnosine. The method further includes cultivating the microorganism in a culture medium until a recoverable quantity of carnosine is produced and recovering the carnosine.

In another aspect of the invention, a method of producing beta-alanine provided. The method comprises providing a recombinant microorganism comprising an engineered beta-alanine biosynthesis pathway. The engineered microorganism may be used for the commercial production of beta-alanine. Accordingly, in one embodiment the invention provides growing in suitable conditions, a recombinant microbial host cell comprising at least one DNA molecule encoding an enzyme that catalyzes an aspartate to beta-alanine conversion (pathway step a) and wherein the at least one DNA molecule is heterologous to said microbial host cell and wherein said microbial host cell produces beta-alanine. The method further includes cultivating the microorganism in a culture medium until a recoverable quantity of beta-alanine is produced and recovering the beta-alanine.

Embodiments of the present invention comprise engineered organisms that produce beta-alanine, carnosine, or both. The engineered organisms may include genetically tractable organisms such as plants, animals, bacteria, or fungi. Some embodiments of the present invention comprise genetically engineered strains of yeast. In further embodiments, the yeast is S. cerevisiae. S. cerevisiae is a preferred organism for biosynthetic production due to favorable consumer sentiment, the robust experience and infrastructure for scaling up fermentation, and lack of potential phage infection.

Strains of the present invention encode enzymes that convert aspartate to beta-alanine, and beta-alanine to carnosine, or a combination thereof.

Compositions of the present invention include yeast strains engineered with heterologous genes to produce beta-alanine and/or carnosine. In one aspect, the engineered organisms have two or three heterologous genes or open reading frames under GAL inducible promoters. In certain embodiments, the heterologous genes are selected from the group consisting of panD, mfnA, amilCP, CARNS1, and ATPGD1. The panD gene encodes the enzyme aspartate decarboxylase which decarboxylates aspartate to produce beta-alanine (pathway step a). In some embodiments, the mfnA gene encodes the enzyme L-tyrosine decarboxylase which decarboxylates aspartate to produce beta-alanine (pathway step a). In some embodiment, beta-alanine is exogenously added to the growth culture and the aspartate decarboxylation step is bypassed. The ATPGD1 gene encodes the enzyme carnosine synthase that catalyzes the joining of beta-alanine to histidine to produce carnosine (pathway step b). In some aspects, the enzyme carnosine synthase is encoded by CARNS1 gene. In another aspect, the engineered organism has a third heterologous gene, panM which encodes a protein that facilitates the maturation of PanD into a functional peptide.

The yeast strains described herein can be used to produce the popular dietary supplements carnosine and beta-alanine via the fermentation of sugars or the biotransformation of aspartate or beta-alanine, or mixtures thereof. The strains may be grown in a bioreactor and will produce carnosine in the cell pellet fraction. The strains may be grown in a bioreactor and will produce beta-alanine. Beta-alanine may be found in the supernatant, cell pellet, or a combination thereof. Subsequently, the carnosine or beta-alanine can be purified and used as a dietary supplement or various other purposes. The strains encode enzymes that convert the native yeast metabolites aspartate to beta-alanine, and/or aspartate and histidine to carnosine via beta-alanine.

The present disclosure provides methods for the biosynthetic production of carnosine and beta-alanine. Embodiments of the present invention comprise growing engineered yeast strains using more generalizable equipment based on fermentation technologies. As a result, the theoretical cost of the biological product could be as low as one-fifth the cost of the existing product, with additional benefits in reducing the capital costs of dedicated facilities, impact on the environment, safety of production workers, and potentially reduced impurities in the final products.

### BRIEF DESCRIPTION OF THE DRAWINGS

FIG. 1 shows the biosynthetic pathway encoded by strains of the present disclosure.

FIG. 2 shows the relative carnosine titer from various strains of the present invention.

FIG. 3 shows the relative beta-alanine titer from various strains of the present invention.

### DETAILED DESCRIPTION OF THE INVENTION

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to those of skill in the art, and are described in numerous standard texts and reference works. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an,” and “the” include the plural reference unless the context clearly indicates otherwise.

Unless otherwise indicated, nucleic acids are written left to right in 5′ to 3′ orientation and amino acid sequences are written left to right in amino to carboxyl orientation, respectively.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the specification as a whole.

The term “invention” or “present invention” as used herein is a non-limiting term and is not intended to refer to any single embodiment of the particular invention but encompasses all possible embodiments as described in the specification and the claims.

A modified microorganism for high efficient production of carnosine and beta-alanine is provided herein. The present disclosure provides compositions and methods for an industrial fermentation process for the production of nutrient supplements such as carnosine and beta-alanine. The fermentation is conducted using various species, including yeast, bacteria, and fungi. The present disclosure also provides compositions and methods for an industrial biotransformation process for the production of supplements such as carnosine. The microorganisms are genetically engineered to produce beta-alanine, carnosine, or both.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

Yeasts are eukaryotic microorganisms classified as members of the fungus kingdom and are estimated to constitute 1% of all described fungal species. Yeasts are unicellular, although some species may also develop multicellular characteristics by forming strings of connected budding cells known as pseudohyphae or false hyphae. Yeasts do not form a single taxonomic or phylogenetic grouping. The term “yeast” is often taken as a synonym for Saccharomyces cerevisiae, but the phylogenetic diversity of yeasts is shown by their placement in two separate phyla: the Ascomycota and the Basidiomycota.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

The term “endogenous gene” refers to a native gene in its natural location in the genome of an organism.

A “foreign gene” or “heterologous gene” refers to a gene not normally found in the host organism, but that is introduced into the host organism by gene transfer. Foreign genes can comprise native genes inserted into a non-native organism, or chimeric genes.

A “transgene” is a gene that has been introduced into the genome by a transformation procedure.

As used herein, the term “open reading frame” also referred to as “ORF” is the part of a reading frame that has the potential to code for a protein or peptide.

As used herein the term “coding sequence” refers to DNA sequence that code for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “genetically engineered” or “genetic engineering” or “genetic modification” involves the direct manipulation of an organism's genome using molecular and biotechnological tools and techniques. The present disclosure relates rational pathway design and assembly of biosynthetic genes, genes associated with operons, and control elements of such nucleic acid sequences, for the production of a desired metabolite, such as carnosine and beta-alanine, in a microorganism.

As used herein, “metabolically engineered” can further include optimization of metabolic flux by regulation and optimization of transcription, translation, protein stability and protein functionality using genetic engineering and appropriate culture condition. The biosynthetic genes can be heterologous to the host (e.g., microorganism), either by virtue of being foreign to the host, or being modified by mutagenesis, recombination, or association with a heterologous expression control sequence in an endogenous host cell. Appropriate culture conditions are conditions such as culture medium pH, ionic strength, nutritive content, etc., temperature, oxygen, CO₂, nitrogen content, humidity, and other culture conditions that permit production of the compound by the host microorganism, i.e., by the metabolic action of the microorganism. Appropriate culture conditions are well known for microorganisms that can serve as host cells.

The term “recombinant microorganism” and “recombinant host cell” are used interchangeably herein and refer to microorganisms that have been genetically modified to express or over-express endogenous polynucleotides, or to express heterologous polynucleotides, such as those included in a vector, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition.

The terms “metabolically engineered microorganism” and “modified microorganism” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The introduction of genetic material into a host or parental microorganism of choice modifies or alters the cellular physiology and biochemistry of the microorganism. Through the introduction of genetic material the parental microorganism acquires new properties, e.g. the ability to produce a new, or greater quantities of, an intracellular metabolite.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a carnosine and/or beta-alanine biosynthetic pathway.

For example, the introduction of genetic material into a parental microorganism results in a new or modified ability to produce a chemical. The genetic material introduced into the parental microorganism contains gene, or parts of genes, coding for one or more of the enzymes involved in a biosynthetic pathway for the production of a chemical and may also include additional elements for the expression or regulation of expression of these genes, e.g. promoter sequences.

Those skilled in the art will understand that the genetic alterations, including metabolic modifications exemplified herein, are described with reference to a suitable host organism such as S. cerevisiae and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the S. cerevisiae metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or non-orthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity.

As used herein, the term “exogenous” or “heterologous” means that a biological function or material, including genetic material, of interest is not natural in a host strain. The term “native” means that such biological material or function naturally exists in the host strain or is found in a genome of a wild-type cell in the host strain.

Exogenous nucleic acid sequences involved in a pathway for production of carnosine and beta-alanine can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that are recognize and bind reacting the protein. See Sambrook et al., 1989, supra.

It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid”, “vector”, and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

As used herein, an “enzymatically active domain” refers to any polypeptide, naturally occurring or synthetically produced, capable of mediating, facilitating, or otherwise regulating a chemical reaction, without, itself, being permanently modified, altered, or destroyed. Binding sites (or domains), in which a polypeptide does not catalyze a chemical reaction, but merely forms noncovalent bonds with another molecule, are not enzymatically active domains as defined herein. In addition, catalytically active domains, in which the protein possessing the catalytic domain is modified, altered, or destroyed, are not enzymatically active domains as defined herein. Enzymatically active domains, therefore, are distinguishable from other (non-enzymatic) catalytic domains known in the art (e.g., detectable tags, signal peptides, allosteric domains, etc.).

The term “homolog”, used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. Thus, the term “homologous proteins” is defined to mean that the two proteins have similar amino acid sequences.

The term “analog” or “analogous” refers to nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

An expression vector or vectors can be constructed to include one or more carnosine and beta-alanine biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art.

When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter.

The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “fermentation” or “fermentation process” is defined as a process in which a microorganism is cultivated in a culture medium containing raw materials, such as feedstock and nutrients, wherein the microorganism converts raw materials, such as a feedstock, into products. Fermentation can be accomplished in batch or continuous production formats.

As used herein, the term “biotransformation” or “bioconversion” is the chemical modification made by an organism on a chemical compound.

As used interchangeably herein, the terms “activity” and “enzymatic activity” refer to any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof.

As used herein, the term “carnosine biosynthesis” refers to a metabolic pathway that produces carnosine. The structure of carnosine is provided herein.

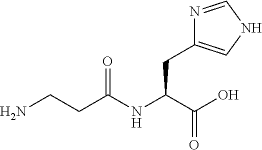 [SMILES: NCCC(=O)N[C@@H](Cc1c[nH]cn1)C(=O)O]

As used herein, the term “beta-alanine biosynthesis” refers to a metabolic pathway that produces beta-alanine. The structure of beta-alanine is provided herein.

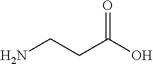

The term “aspartate decarboxylase” refers to an enzyme that catalyzes the conversion of aspartate to beta-alanine. These enzymes are available from a vast array of organisms. The enzyme may be, for example, encoded by the panD gene from Corynebacterium glutamicum, Escherichia coli, Helicobacter pylori, Tribolium castaneum, Pectobacterium carotovorum, Actinoplanes sp. SE50/110, or Taoultella ornithinolytica. The enzyme may be, for example encoded by the mfnA gene from Methanocaldococcus jannaschii DSM 2661 or Methanocaldococcus bathoardescens.

The term “carnosine synthase” refers to an enzyme that catalyzes the joining of beta-alanine to histidine to produce carnosine. These enzymes are available from a vast array of organisms. The enzyme may be, for example, encoded by the ATPGD1 gene from Gallus gallus, or CARNS1 gene from Gorilla gorilla, Falco perefrinus, Allpiucator mississsippiensis, Ailuoropoda melanoleuca, Ursus maritimus, Python bivittatus, or Orcinus orca.

The term “PanD autocleavage accelerator” refers to a polypeptide that facilitates the maturation of PanD into the functional peptide. This polypeptide is available from a vast array of organisms, for example, Escherichia coli.

The first step (pathway step a) in carnosine and beta-alanine biosynthesis is the direct decarboxylation of aspartate to beta-alanine which is catalyzed by an aspartate decarboxylase. This may be encoded by the panD gene from Corynebacterium glutamicum Escherichia coli, Helicobacter pylori, Tribolium castaneum, Pectobacterium carotovorum, Actinoplanes sp. SE50/110, or Taoultella ornithinolytica. The enzyme may be, for example encoded by the mfnA gene from Methanocaldococcus jannaschii DSM 2661 or Methanocaldococcus bathoardescens. Beta-alanine may be purified after this step.

In the second step (pathway step b), beta-alanine is joined with another amino acid, histidine, to produce carnosine. This is catalyzed by carnosine synthase which may be encoded by ATPGD1 gene from Gallus gallus, or CARNS1 gene from Gorilla gorilla, Falco perefrinus, Allpiucator mississsippiensis, Ailuoropoda melanoleuca, Ursus maritimus, Python bivittatus, or Orcinus orca.

Alternatively, pathway step “a” may be bypassed by exogenous addition of beta-alanine to the growth medium thereby enabling production of carnosine by the biotransformation of alanine via carnosine synthase.

Strains of the present invention may comprise a PanD autocleavage accelerator to facilitate maturation of PanD enzyme. This enzyme is available from a vast array of organisms. The enzyme may be, for example, the panM gene from Escherichia coli.

The term “volumetric productivity” or “production rate” is defined as the amount of product formed per volume of medium per unit of time. Volumetric productivity is reported in gram per liter per hour (g/L/h).

The term “yield” is defined as the amount of product obtained per unit weight of raw material and may be expressed as grams product per grams substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titer” is defined as the concentration of a substance in solution. Herein, it also refers to the concentration of product, usually expressed in grams per liter (g/L), upon completion of fermentation.

### Construction of Production Host

Recombinant organisms containing the necessary genes that will encode the enzymatic pathway for the biosynthetic production of beta-alanine and carnosine may be constructed using techniques well known in the art. In the present invention, genes encoding the enzymes of one of the carnosine and beta-alanine biosynthetic pathways of the invention, for example, aspartate decarboxylase and carnosine synthase may be determined from the genomes of various organisms, as described above.

Methods of obtaining desired genes from a genome are common and well known in the art of molecular biology. For example, if the sequence of the gene is known, suitable synthetic genes are constructed by gene synthesis. Tools for codon optimization for expression in a heterologous host are readily available.

Once the relevant pathway genes are identified, the synthesized genes may be assembled into larger genetic constructs such as into suitable vectors. Means for this are well known in the art. Vectors or cassettes useful for the transformation of a variety of host cells are common and commercially available from gene synthesis companies such as DNA2.0, SGI-DNA, Invitrogen, and Genscript. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

### Engineered Microorganisms

According to one embodiment, a modified microorganism comprising a heterologous production system of carnosine and beta-alanine is provided. The modified microorganisms may be yeast, bacteria, or fungi. The modified microorganisms may express heterologous proteins useful in the production of beta-alanine and/or carnosine.

One embodiment of the present invention is a non-naturally occurring microorganism having a beta-alanine pathway and comprising at least one exogenous one open reading frame encoding an enzyme expressed in a sufficient amount to produce beta-alanine, and wherein said enzyme converts aspartate to beta-alanine (pathway step a).

One embodiment of the present invention is a non-naturally occurring microorganism having a carnosine synthesis pathway and comprising at least one exogenous one open reading frame encoding an enzyme expressed in a sufficient amount to produce carnosine, and wherein said enzyme converts exogenously added beta-alanine to carnosine (pathway step b).

Yet another embodiment of the present invention is a non-naturally occurring microorganism having a carnosine biosynthesis pathway comprising at least two open reading frames encoding carnosine pathway enzymes expressed in a sufficient amount to produce carnosine, wherein said pathway comprises (i) aspartate to beta-alanine (pathway step a) and (ii) beta-alanine to carnosine (pathway step b).

In another embodiment, the present invention provides a non-naturally occurring microorganism having a carnosine and beta-alanine pathway and comprising at least three open reading frames encoding carnosine and beta-alanine pathway enzymes expressed in a sufficient amount to produce carnosine and beta-alanine, wherein said carnosine and beta-alanine pathway comprises (i) aspartate to beta-alanine (pathway step a) and (ii) beta-alanine to carnosine (pathway step b).

In some embodiments of the present invention, the enzyme that converts aspartate to beta-alanine is an aspartate decarboxylase. In some embodiments of the present invention, the enzyme that converts aspartate to beta-alanine is an L-tyrosine/L-aspartate decarboxylase. In other embodiments of the present invention, the enzyme that converts beta-alanine to carnosine is carnosine synthase. In other embodiments of the present invention, the enzyme that converts beta-alanine to carnosine is chromoprotein. In yet other embodiments of the present invention, the microorganisms comprise a PanD autocleavage accelerator such as that encoded by the panM gene from E. coli.

Examples of exogenous genes that may be expressed in modified microorganisms of the present invention include genes that encode enzymes such as aspartate decarboxylase, carnosine synthase, and PanD autocleavage accelerator. These genes may be derived from animals, plants, bacteria, yeast, or fungi. Further, said nucleic acid encoding molecules (e.g., genes) may be codon optimized for use in an organism of interest.

In some embodiments, the modified microorganism is a yeast cell. In some embodiments, the recombinant microorganisms may be yeast recombinant microorganisms of the Saccharomyces clade. In certain embodiments, the modified yeast may be Saccharomyces cerevisiae. The S. cerevisiae may be strain S288C or a derivative thereof. The modified yeast may encode at least one heterologous enzyme selected from the group consisting of aspartate decarboxylase, carnosine synthase, and PanD autocleavage accelerator. The heterologous genes encoding these enzymes may be derived from bacteria, yeast, fungi, plants, or animals. The aspartate decarboxylase may be a Corynebacterium glutamicum panD gene and encode a polypeptide comprising SEQ ID NO: 1 or the active domain thereof. The aspartate decarboxylase may be an Escherichia coli panD gene and encode a polypeptide comprising SEQ ID NO: 12 or the active domain thereof. The aspartate decarboxylase may be a Helicobacter pylori panD gene and encode a polypeptide comprising SEQ ID NO: 14 or the active domain thereof. The aspartate decarboxylase may be a Tribolium castaneum panD gene and encode a polypeptide comprising SEQ ID NO: 15 or the active domain thereof. The aspartate decarboxylase may be a Pectobacterium carotovorum panD gene and encode a polypeptide comprising SEQ ID NO: 17 or the active domain thereof. The aspartate decarboxylase may be an Actinoplanes sp. SE50/110 panD gene and encode a polypeptide comprising SEQ ID NO: 18 or the active domain thereof. The aspartate decarboxylase may be a Raoultella ornithinolytica panD gene and encode a polypeptide comprising SEQ ID NO: 19 or the active domain thereof. The aspartate dexarboxylase may be L-tyrosine/L-aspartate decarboxylase. The L-tyrosine/L-aspartate decarboxylase may be a Methanocaldococcus jannaschii DSM 2661 mfnA gene and encode a polypeptide comprising SEQ ID No: 13 or the active domain thereof. The aspartate decarboxylase may be L-tyrosine decarboxylase from a Methanocaldococcus bathoardescens mfnA gene and encode a polypeptide comprising SEQ ID No: 16 or the active domain thereof. The carnosine synthase may be a Gallus gallus ATPGD1 gene and encode a polypeptide comprising SEQ ID NO: 2 or the active domain thereof. The carnosine synthase may be a Gorilla gorilla CARNS1 gene and encode a polypeptide comprising SEQ ID NO: 5 or the active domain thereof. The carnosine synthase may be a Falco peregrinus CARNS1 gene and encode a polypeptide comprising SEQ ID NO: 6 or the active domain thereof. The carnosine synthase may be an Alligator mississippiensis CARNS1 gene and encode a polypeptide comprising SEQ ID NO: 7 or the active domain thereof. The carnosine synthase may be an Ailuropoda melanoleuca CARNS1 gene and encode a polypeptide comprising SEQ ID NO: 8 or the active domain thereof. The carnosine synthase may be an Ursus maritimus CARNS1 gene and encode a polypeptide comprising SEQ ID NO: 9 or the active domain thereof. The carnosine synthase may be a Python bivittatus CARNS1 gene and encode a polypeptide comprising SEQ ID NO: 10 or the active domain thereof. The carnosine synthase may be an Orcinus orca CARNS1 gene and encode a polypeptide comprising SEQ ID NO: 11 or the active domain thereof. The PanD autocleavage accelerator may be an Escherichia coli panM gene and encode a polypeptide comprising SEQ ID NO: 3 or the active domain thereof.

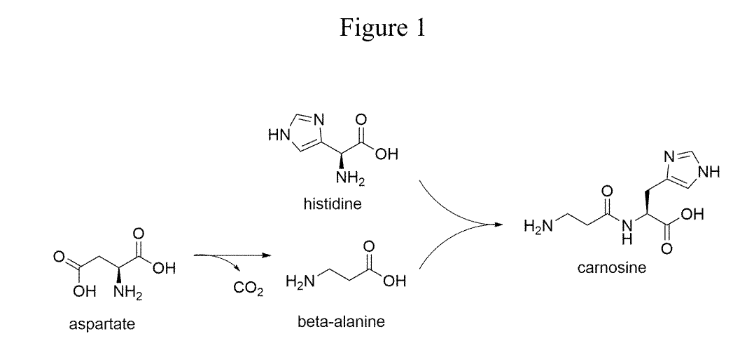

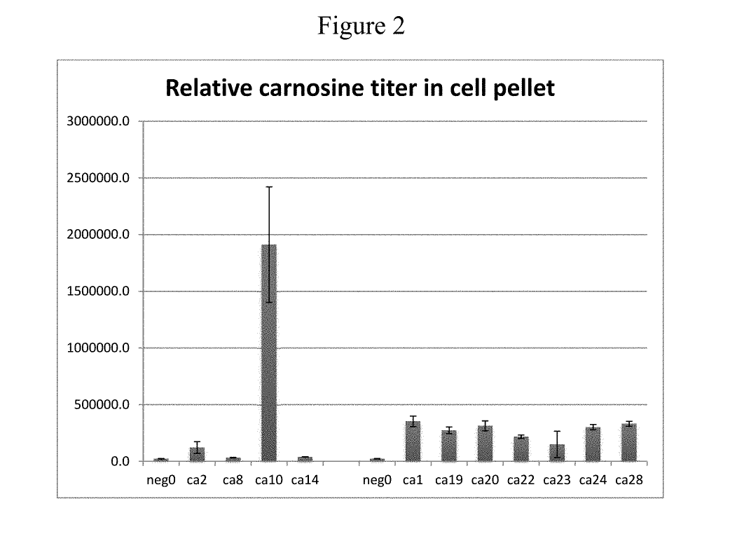

The biosynthetic pathway encoded by these strains is described in FIG. 1. The amino acid aspartate is decarboxylated to beta-alanine via the action of aspartate decarboxylase. Beta-alanine is then joined with histidine, to produce carnosine via carnosine synthase. Strain ca1 differs from ca2 by the addition of the Escherichia coli panM encoding a PanD autocleavage accelerator. This protein is expected to facilitate the maturation of PanD into the functional peptide. Indeed, higher production of carnosine in strain ca1 compared to strain ca2 was observed (See FIG. 2). When grown in SC Minimal Broth with 2% raffinose and 1% galactose, strains ca1 produces carnosine in cell pellets, as indicated by LC-MS analysis. No detectable accumulation of carnosine was observed in the supernatant.

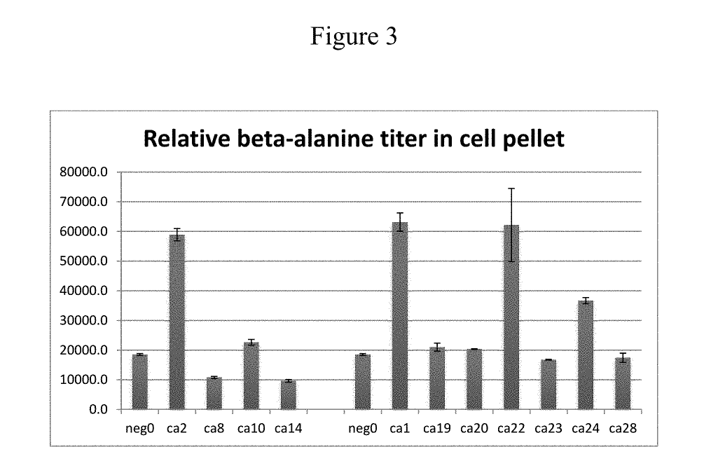

The relative activities of ortholog variants are described in FIG. 2, FIG. 3 and Table 2. Strains ca8-ca17 exchange the Gallus gallus ATPGD1 gene in ca2 with orthologs from other organisms. Of these, ca8 (Gorilla gorilla), ca10 (Alligator mississippiensis), and ca14 (Python bivittatus) show higher production of carnosine than ca2 with 16× improvement with the Alligator carnosine synthase (ca10, FIG. 2). To confirm that the carnosine-formation reaction is carried out by CARNS1 and not an endogenous enzyme, a variant encoding an unrelated protein, amilCP, was included in place of the carnosine synthase. In strain ca7, the carnosine synthase allele is replaced by the unrelated chromoprotein amilCP. Omitting this activity resulted in undetectable production of carnosine. Strains ca19-ca28 exchange the Corynebacterium glutamicum panD gene in ca1 with orthologs. The panD variants show comparable or lower carnosine and beta-alanine titers than ca1 (FIGS. 2 and 3).

### Methods of Production

The present disclosure provides methods for the biosynthetic production of beta-alanine and carnosine using engineered microorganisms of the present invention.

In one embodiment, a method of producing beta-alanine is provided. The method comprises providing a fermentation media comprising a carbon substrate, contacting said media with a recombinant yeast microorganism expressing an engineered beta-alanine biosynthetic pathway wherein said pathway comprises an aspartate to beta-alanine conversion (pathway step a), and culturing the yeast in conditions whereby beta-alanine is produced.

In another embodiment of the present invention, a method of producing carnosine is provided. The method comprises providing a fermentation media comprising a carbon substrate, contacting said media with a recombinant yeast microorganism expressing an engineered carnosine biosynthetic pathway wherein said pathway comprises (i) an aspartate to beta-alanine conversion (pathway step a) and (ii) a beta-alanine to carnosine conversion (pathway step b), and culturing the yeast in conditions whereby carnosine is produced.

In another embodiment of the present invention, a method of producing carnosine via biotransformation is provided. The method comprises providing a media comprising a carbon substrate and exogenously added beta-alanine, contacting said media with a recombinant yeast microorganism expressing an engineered carnosine biosynthetic pathway wherein said pathway comprises (i) a beta-alanine to carnosine conversion (pathway step b), and culturing the yeast in conditions whereby carnosine is produced.

Some embodiments of the present invention comprise yeast strains designated ca1 and ca2 and are derived from S. cerevisiae strain S288C. Each encodes at least 2 foreign genes under inducible Gal promoters. Strain ca1 also contains an additional gene, panM. The specific proteins encoded by each strain and their sequences, source, and accession numbers are provided in Table 1. The genes for these proteins are synthesized with yeast-optimized codon usage, assembled into singular genetic cassettes, and then inserted into the HO locus of S288C under URA2 selection. Strains ca1 and ca2 served as parent strains to derivatives comprising various heterologous genes. Ca2 served as a parent strain for ca7, ca8, ca9, ca10, ca11, ca12, ca14, ca15 in which the carnosine synthase is a different ortholog. Strain ca1 served as the parent strain to strains ca19, ca20, ca21, ca22, ca23, ca24, ca27, and ca28 in which the aspartate decarboxylase is a different ortholog. The specific proteins encoded by each strain and their sequences, source, and accession numbers are provided in Table 2.

Aspartate, histidine, and the cofactors involved in the carnosine and beta-alanine pathway are universal to all organisms, and thus the host organism could be any genetically tractable organism (plants, animals, bacteria, or fungi). Amongst yeasts, other species such as S. pombe or P. pastoris are plausible alternatives. Within the S. cerevisiae species, other strains more amenable to large-scale productions, such as CENPalpha, may be utilized.

The Gal promoter used in embodiments of the present invention could be replaced with constitutive promoters, or other chemically-inducible, growth phase-dependent, or stress-induced promoters. Heterologous genes of the present invention may be genomically encoded or alternatively encoded on plasmids or yeast artificial chromosomes (YACs). All genes introduced could be encoded with alternate codon usage without altering the biochemical composition of the system. All enzymes used in embodiments of the present invention have extensive orthologs in the biosphere that could be encoded as alternatives.

Aspartate, histidine, and the cofactors involved in this pathway are universal to all organisms, and thus the host organism could be any genetically tractable organism (plants, animals, bacteria, or fungi). Among yeast, other species such as S. pombe or P. pastoris are plausible alternatives. Within the S. cerevisiae species, other strains more amenable to large scale productions, such as CENPalpha, may be preferable. The panD gene can replaced with orthologs from other bacteria. Examples include Corynebacterium glutamicum Escherichia coli, Helicobacter pylori, Tribolium castaneum, Pectobacterium carotovorum, Actinoplanes sp. SE50/110, Taoultella ornithinolytica, Methanocaldococcus jannaschii DSM 2661 and Methanocaldococcus bathoardescens. This is shown in Table 2. Carnosine synthase is natively found in mammals, birds, and reptiles. Therefore, the chicken enzyme used in ca1 and ca2 could be replaced by various orthologs. Examples include Gorilla gorilla, Falco perefrinus, Allpiucator mississsippiensis, Ailuoropoda melanoleuca, Ursus maritimus, Python bivittatus, and Orcinus orca. This is shown in Table 2.

### Culture Conditions

The growth medium used to test for production of carnosine by the engineered strains was Teknova SC Minimal Broth with Raffinose supplemented with 1% galactose.

A variety of purification protocols including solid phase extraction and cation exchange chromatography may be employed to purify the desired products from the culture supernatant or the yeast cell pellet fraction.

### Examples

### Example 1: Strain Development

Two yeast prototypes constructed and successfully tested (strains ca1 and ca2) are derived from S. cerevisiae strain S288C. Each encodes 2 or 3 genes under inducible Gal promoters. The specific proteins encoded by each strain and their sequences, source, and accession numbers are provided in Table 1. The genes for these proteins were synthesized with yeast optimized codon usage, assembled into singular genetic cassettes, and then inserted into the HO locus of S288C under URA2 selection.

TABLE 1

| | Accession | | | |
|---|---|---|---|---|
| Strain | No. | Source | Name | Enzyme |
| ca1 | D3KCC4 | Gallus gallus | ATPGD1 | carnosine synthase |
| | Q9X4N0 | Corynebacterium | panD | aspartate 1- |
| | | glutamicum | | decarboxylase |
| | CQR82874 | Escherichia coli | panM | PanD autocleavage |
| | | | | accelerator |
| ca2 | D3KCC4 | Gallus gallus | ATPGD1 | carnosine synthase |
| | Q9X4N0 | Corynebacterium | panD | aspartate 1- |
| | | glutamicum | | decarboxylase |

TABLE 2
Ortholog Variants of ca1 and ca2

| | Parent | Accession | | | |
|---|---|---|---|---|---|
| Strain | Strain | No. | Source | Name | Enzyme |
| ca7 | ca2 | AAU06854 | Acropora millepora | amilCP | Chromoprotein |
| ca8 | ca2 | XP_004051679 | Gorilla gorilla | CARNS1 | carnosine |
| | | | | | synthase 1 |
| | | | | | isoform X2 |
| ca9 | ca2 | XP_013159432 | Falco peregrinus | CARNS1 | carnosine |
| | | | | | synthase 1 |
| ca10 | ca2 | XP_006260145 | Alligator | CARNS1 | carnosine |
| | | | mississippiensis | | synthase 1 |
| ca11 | ca2 | XP_011225873 | Ailuropoda | CARNS1 | carnosine |
| | | | melanoleuca | | synthase 1 |
| ca12 | ca2 | XP_008707395 | Ursus maritimus | CARNS1 | carnosine |
| | | | | | synthase 1 |
| ca14 | ca2 | XP_007425192 | Python bivittatus | CARNS1 | carnosine |
| | | | | | synthase 1 |
| ca15 | ca2 | XP_004278053 | Orcinus orca | CARNS1 | carnosine |
| | | | | | synthase 1 |
| ca19 | ca1 | WP_000621503 | Escherichia coli | panD | aspartate 1- |
| | | | | | decarboxylase |
| ca20 | ca1 | Q60358 | Methanocaldococcus | mfnA | L-tyrosine/L- |
| | | | jannaschii DSM 2661 | | aspartate |
| | | | | | decarboxylase |
| ca21 | ca1 | WP_000142250 | Helicobacter pylori | panD | aspartate 1- |
| | | | | | decarboxylase |
| ca22 | ca1 | NP_001096055 | Tribolium castaneum | panD | aspartate 1- |
| | | | | | decarboxylase |
| ca23 | ca1 | WP_048201473 | Methanocaldococcus | mfnA | L-tyrosine |
| | | | bathoardescens | | decarboxylase |
| ca24 | ca1 | WP_039493424 | Pectobacterium | panD | aspartate 1- |
| | | | carotovorum | | decarboxylase |
| ca27 | ca1 | WP_014694997 | Actinoplanes sp. | panD | aspartate 1- |
| | | | SE50/110 | | decarboxylase |

The biosynthetic pathway encoded by these strains is described in FIG. 1. The amino acid aspartate is decarboxylated to beta-alanine via the action of aspartate decarboxylase. This step may be bypassed by the exogenous addition of Beta-alanine. Beta-alanine is then joined with another amino acid, histidine, to produce carnosine via the carnosine synthase. Strain ca1 and ca2 differ by the inclusion of a third gene in ca1, panM from E. coli. This protein facilitates the maturation of PanD into the functional peptide.

### Example 2: Carnosine and Beta-Alanine Production

To test strains for chemical production, cells were grown in medium and then prepared for analysis by LC-MS. Medium containing 2% raffinose minus uracil from Teknova was prepared according to the manufacturer's protocol and is referred to as “Pregrowth Medium”. The same medium supplemented with 1% galactose was prepared as “Induction Medium”. Plastic 24-well plates were filled with 3 mL of Pregrowth Medium and then inoculated with frozen yeast stocks. The blocks were grown with shaking at 30° C. for 48 hours to generate saturated pregrowth cultures. These cultures were diluted 10 L into 4 mL of Induction Medium in additional 24-well plates to induce expression of the expressed genes. In some experiments, beta-alanine, histidine, or aspartate were also included in the induction culture. The plates were grown with shaking at 30° C. for 48 hours to generate saturated induction cultures. The plates were then subjected to centrifugation at 6000 rcf for 5 min to pellet the cells. Aliquots of clarified supernatant were transferred to a 96-well plate for analysis by LC-MS. The cells were then centrifuged a second time and the remainder of the supernatant removed. To prepare pellet extracts, 1 mL of room temperature methanol was added to each well and the cells were resuspended by shaking for 5 min. The plate was again centrifuged to remove cell debris, and the clarified extract was transferred to a 96-well plate. The collected samples were analyzed in 2 microliter aliquots by LC-MS on a Waters Xevo-G2-XS-QT of with a C18 column and a mobile phase gradient between 0.1% formic acid and acetonitrile with 0.1% formic acid. Two technical replicates of the induction, extraction, and analysis steps were performed for each experimental condition.

1.-23. (canceled)

24. A modified microorganism for production of carnosine and beta-alanine comprising at least one heterologous enzyme selected from the group consisting of aspartate decarboxylase, carnosine synthase, and PanD autocleavage accelerator.

25. The modified microorganism of claim 24 wherein the aspartate decarboxylase comprises an amino acid sequence selected from the group consisting of SEQ ID NO: 1, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or the active domain thereof.

26. The modified microorganism of claim 24 wherein the carnosine synthase comprises an amino acid sequence selected from the group consisting of SEQ ID NO: 2, SEQ ID NO: 5, SEQ ID NO: 6, SEQ ID NO: 7, SEQ ID NO: 8, SEQ ID NO: 9, SEQ ID NO: 10, SEQ ID NO: 11, or the active domain thereof.

27. The modified microorganism of claim 25 wherein the carnosine synthase comprises an amino acid sequence selected from the group consisting of SEQ ID NO: 2, SEQ ID NO: 5, SEQ ID NO: 6, SEQ ID NO: 7, SEQ ID NO: 8, SEQ ID NO: 9, SEQ ID NO: 10, SEQ ID NO: 11, or the active domain thereof.

28. The modified microorganism of claim 24 wherein the PanD autocleavage accelerator comprises SEQ ID NO: 3 or the active domain thereof.

29. The modified microorganism of claim 25 wherein the PanD autocleavage accelerator comprises SEQ ID NO: 3 or the active domain thereof.

30. The modified microorganism of claim 26 wherein the PanD autocleavage accelerator comprises SEQ ID NO: 3 or the active domain thereof.

31. The modified microorganism of claim 24, wherein the modified microorganism is yeast or bacteria.

32. The modified microorganism of claim 31, wherein the yeast is Saccharomyces cerevisiae strain S288C.

33. A method of producing carnosine comprising: (a) culturing the cells of claim 24 under suitable conditions for the production of carnosine; (b) producing carnosine; and (c) recovering the carnosine.

34. The method of claim 33 wherein recovering the carnosine or beta-alanine comprises isolating the carnosine or beta-alanine from the supernatant, the cell pellet, or a combination thereof.

35. A non-naturally occurring microbial organism having a carnosine pathway and comprising at least one heterologous enzyme expressed in a sufficient amount to produce carnosine, wherein said carnosine pathway comprises (i) an enzyme that converts aspartate to beta-alanine and (ii) an enzyme that converts beta-alanine to carnosine.

36. The non-naturally occurring microbial organism of claim 35 wherein said enzyme that converts aspartate to beta-alanine is an aspartate decarboxylase and wherein said enzyme that converts beta-alanine to carnosine is a carnosine synthase.

37. The non-naturally occurring microbial organism of claim 36 wherein said aspartate decarboxylase comprises an amino acid sequence selected from the group consisting of SEQ ID NO: 1, SEQ ID NO: 12, SEQ ID NO: 14, SEQ ID NO: 15, SEQ ID NO: 17, SEQ ID NO: 18, and SEQ ID NO: 19, and said carnosine synthase comprises an amino acid sequence selected from the group consisting of SEQ ID NO: 2, SEQ ID NO: 5, SEQ ID NO: 6, SEQ ID NO: 7, SEQ ID NO: 8, SEQ ID NO: 9, SEQ ID NO:10, and SEQ ID NO:11.

38. The non-naturally occurring microbial organism of claim 37 where said aspartate decarboxylase comprises the amino acid sequence of SEQ ID NO: 1 and wherein said carnosine synthase comprises the amino acid of SEQ ID NO: 7.

39. The non-naturally occurring microbial organism of claim 34 wherein said enzyme that converts aspartate to beta-alanine is an L-tyrosine/L-aspartate decarboxylase and wherein said enzyme that converts beta-alanine to carnosine is a carnosine synthase.

40. The non-naturally occurring microbial organism of claim 39 wherein said L-tyrosine/L-aspartate decarboxylase comprises an amino acid sequence selected from the group consisting of SEQ ID No: 13 and SEQ ID NO: 16 and wherein said carnosine synthase comprises an amino acid sequence selected from the group consisting of SEQ ID NO: 2, SEQ ID NO:5, SEQ ID NO: 6, SEQ ID NO: 7, SEQ ID NO: 8, SEQ ID NO: 9, SEQ ID NO:10, and SEQ ID NO: 11.

41. The non-naturally occurring microbial organism of claim 35 further comprising a PanD autocleavage accelerator wherein said accelerator comprises SEQ ID NO: 3.

42. The non-naturally occurring microbial organism of claim 36 further comprising a PanD autocleavage accelerator wherein said accelerator comprises SEQ ID NO: 3.

43. The non-naturally occurring microbial organism of claim 37 further comprising a PanD autocleavage accelerator wherein said accelerator comprises SEQ ID NO: 3.
